(19) United States
(12) Patent Application Publication
(10) Pub. No.: US 2008/0097405 A1
(43) Pub. Date: Apr. 24, 2008

(54) Luer fitting for power injectable PICC

(76) Inventor: Stephen C. Miller, Queensbury, NY (US)

Correspondence Address: FAY KAPLUN & MARCIN, LLP, 15O BROADWAY, SUITE 702, NEW YORK, NY, 10038, US

(21) Appl. No.: 11/495,449

(22) Filed: Jul. 28, 2006

(51) Int. Cl. A61M 25/18

(52) U.S. Cl. 604/533

(57) ABSTRACT

A fitting for connecting a catheter to a fluid conduit, comprises a housing defining a lumen extending therethrough from a distal end adapted for attachment to a catheter and a proximal end releasably connectable to a second fitting in combination with a wing extending from the housing away from an axis of the lumen, a portion of the housing opposite the wing being substantially rounded and free of projections away from the axis, a radially outermost lateral edge of a first surface of the wing being rounded wherein, when in a desired position, the first surface faces the skin.

### BACKGROUND OF THE INVENTION

The treatment of chronic diseases often involves repeated and prolonged access to the vascular system for the introduction and/or withdrawal of fluids making it impractical and potentially hazardous to insert and remove a catheter from a vessel access at every session. Thus, semi-permanent catheters have been employed to facilitate repeated access to the vascular system while reducing discomfort and side effects. One such catheter, a Peripherally Inserted Central Catheter (PICC), enters the body via a peripheral vein through which it is advanced through a vessel to the heart.

High pressure power injection procedures are used for certain applications such as, for example, the injection of contrast media used for visualization. Catheters used for such procedures are selected to withstand the high pressure without damage while the connections to such catheters are adapted to remain attached and resist leaking while passing the high pressure fluid.

Conventional luer fittings used for power injection procedures have geometry that generally comprise two symmetrical “wings” or other protrusions to give the user sufficient leverage during the connection and disconnection of the luer fittings to obtain the tight fit necessary to effectively seal at the high pressures to which the connection will be exposed. As shown in FIG. 1, a typical luer fitting 100 defines a lumen extending therethrough and includes a pair of substantially planar wings 102 extending in a plane including an axis of the luer fitting 100. The fitting 100 extends from the proximal end of a fluid conduit 106 and comprises a connection portion 104 which may be coupled to and disconnected from a corresponding luer fitting on the distal end of a further device (e.g., a fluid conduit to an external fluid source or destination) as desired to form a fluid connection between the lumen 101 and the further device. To form or remove the connection between fitting 100 and a luer fitting of a further device, the user grasps the wings 102 of the fitting 100 (and corresponding structures of the luer fitting of the further device) and twists the fittings relative to one another while pushing or pulling. When in place on the skin, the fitting 100 is supported by a central portion of the fitting 100 and at least one of the wings 102. The wings 102 protrude laterally so that outer edges thereof are separated by a width W which may, for example, be approximately 0.50 inches while a length L of the wings 102 is, for example, 0.575 inches.

### SUMMARY OF THE INVENTION

In one aspect, the present invention is related to a fitting for connecting a catheter to a fluid conduit, comprises a housing defining a lumen extending therethrough from a distal end adapted for attachment to a catheter and a proximal end releasably connectable to a second fitting in combination with a wing extending from the housing away from an axis of the lumen, a portion of the housing opposite the wing being substantially rounded and free of projections away from the axis, a radially outermost lateral edge of a first surface of the wing being rounded wherein, when in a desired position, the first surface faces the skin.

### BRIEF DESCRIPTION OF THE DRAWINGS

FIG. 1 is a perspective view of a conventional luer fitting;

FIG. 2 is a perspective view of a luer fitting according to an embodiment of the present invention;

FIG. 3 is a front view of the luer fitting shown in FIG. 2;

FIG. 4 is a perspective view showing two luer fittings in parallel, according to embodiments of the present invention;

FIG. 5 is a perspective view showing a second embodiment of a luer fitting according to the invention;

FIG. 6 is a perspective view showing a third embodiment of a luer fitting according to the invention;

FIG. 7 is a perspective view showing another embodiment of a luer fitting according to the invention;

FIG. 8 is a top plan view of the luer fitting shown in FIG. 7;

FIG. 9 is a top view of two luer fittings according to the present invention side by side with one another laying over two conventional luer fittings side by side with one another;

FIG. 10 is an end view of the luer fittings of FIG. 9; and

FIG. 11 is a perspective view of the luer fitting of FIG. 6.

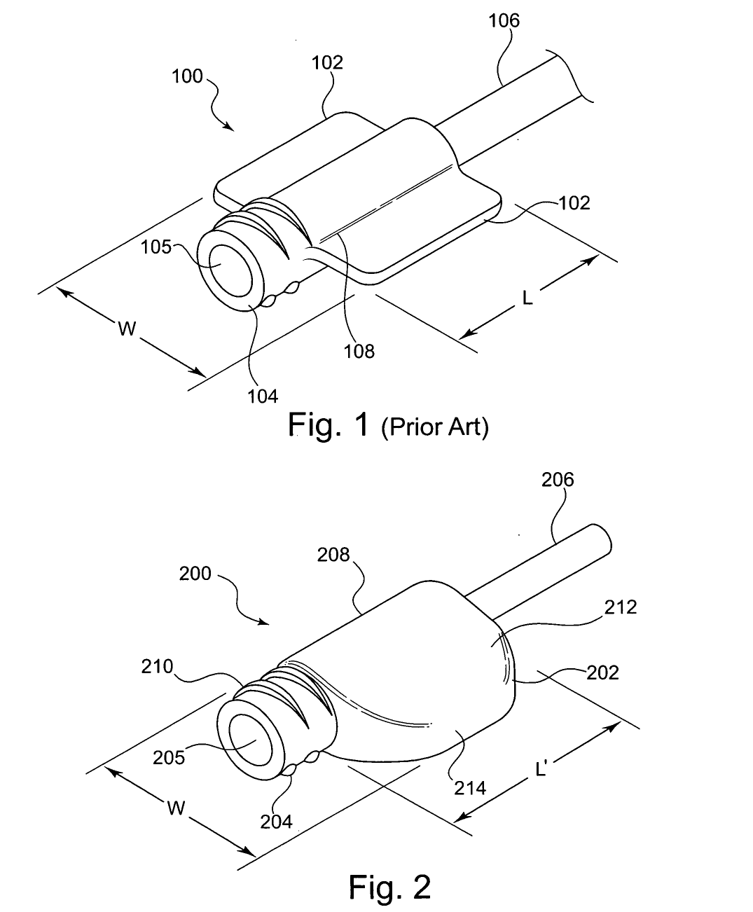

### DETAILED DESCRIPTION

The present invention may be further understood with reference to the following description and the appended drawings, wherein like elements are referred to with the same reference numerals. The invention relates to devices for connecting high pressure fluid sources to catheters and, more specifically, relates to luer fittings for coupling peripherally inserted central venous catheters (PICC) to sources of fluids for introduction at high flow rates and/or pressures.

The luer fittings according to the exemplary embodiments of the present invention are shaped to increase comfort while facilitating leak-free connections even when exposed to high pressures such as those employed in power injections.

An exemplary embodiment of a luer fitting according to the present invention is shown in FIG. 2. The luer fitting 200 defines a fluid lumen 205 which, when the fitting 200 is attached to a fluid conduit 206 of a separate device in a conventional manner, fluidly couples a distal end of the conduit 206 to the lumen 205. Those skilled in the art will understand that the conduit 206 may, for example, be a catheter such as a PICC catheter adapted for use in power injection procedures. The luer fitting 200 comprises a connection portion 204 forming a fluid tight connection with a corresponding connection portion of a second luer fitting of another medical device. The connection portion 204 may have threads, lugs or other mechanical features to engage the second luer fitting as would be understood by those skilled in the art. In a common implementation, the two luer fittings are pushed together and rotated about a common longitudinal axis to form the connection. To release the connection, the luer fittings are rotated in the opposite direction, then pulled apart.

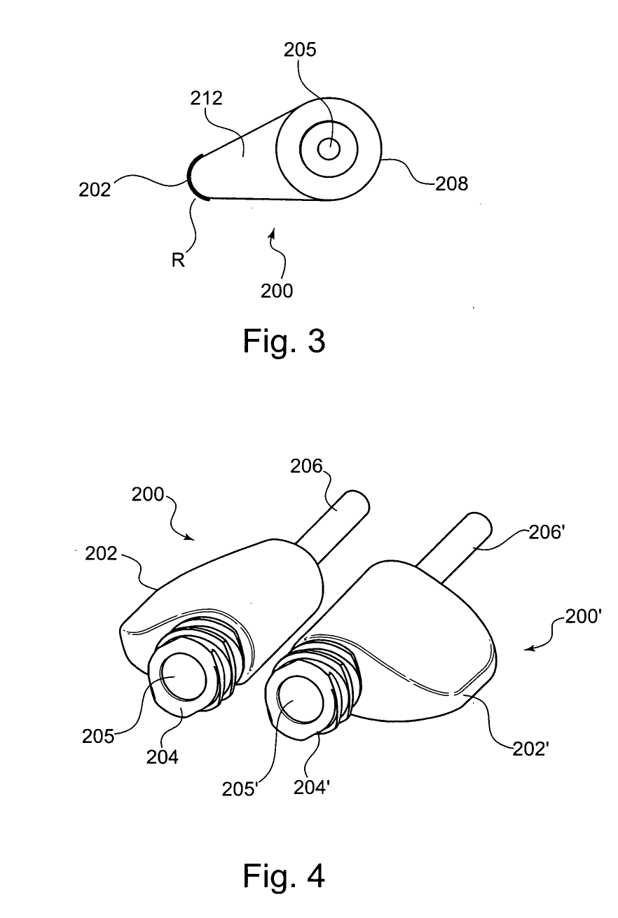

When the luers are used for power injection procedures, a significant amount of force may be necessary to push the fittings together and rotate them into a sealing relationship sufficient to withstand the pressures. As described above, it is important to form the luer fitting so that a user may easily apply force sufficient to engage and detach the connection. According to the invention, the exemplary luer fitting 200 comprises a single “wing” or protrusion 202 extending from one side of a housing 208 of the device. Lateral edges (e.g., edges extending along a radially outermost portion of the wing 202) of skin contacting surfaces are all rounded and free of sharp edges. As shown in FIG. 3, a curved surface with a radius R joins the surfaces of the protrusion 202. The radius R is preferably at least 0.015 inches and more preferably at least 0.030 inches. Those skilled in the art will understand that, for fittings 200 that are symmetric and designed for placement with either of the surfaces 212 facing the skin, all of the lateral edges will be rounded while asymmetric fittings 200 designed for placement in only one orientation may include rounded lateral edges only on those surfaces which will face the skin. The shape and dimensions of the protrusion 202 are selected to provide a lever arm sufficiently long that a user may easily generate an amount of torque sufficient to easily attach and detach the luer fitting 200 from the corresponding second fitting. For example, a length L′ of the protrusion 202 may be between approximately 0.40 and approximately 0.80 inches while a width of the protrusion 202 is between approximately 0.45 and approximately 0.50 inches. The protrusion 202 may more preferably extend approximately 0.60 inches between the distal and proximal ends with a lateral extent of approximately 0.476 inches from a laterally outer-most portion of the protrusion 202 to a laterally opposite side of the housing 208. Those skilled in the art will understand that these dimensions are exemplary only and that any dimension that provides sufficient leverage to enable the user to achieve and release the connection will be suitable.

The protrusion 202 comprises first and second flat surfaces 212 connected to one another by curved edges 214. This removes all sharp edges and abrupt shape changes from the surfaces of the luer fitting 200 which will be in contact with the skin. Both of the flat surfaces 212 lie flat on the skin and, together with the curved surfaces 214, present an overall surface profile free of pressure points that may cause discomfort and/or other complications such as sores and resulting infections, etc. In addition, the relatively large flat surfaces 212 are well suited to receive printed materials, molded information or other types of graphics thereon such as, for example, instructions for use, safety notices, brand information, etc.

FIG. 3 shows a different view of the luer fitting 200, depicting additional flat surfaces 213 on the ends of the fitting 200 which are also available to receive graphics. The protrusion 202 is sized and shaped to obviate the need for a separate balancing ‘wing’ on the opposite side of the lumen 205 as in conventional luers, while giving the user a mechanical advantage to facilitate the application of torque sufficient to lock and unlock the fitting 200. In the exemplary embodiment, the protrusion 202 combines with the housing 208 to form a generally wedge shaped element that is easily grasped and which, as described above, provides a lever to facilitate rotation about the axis of the lumen 205 in locking and unlocking the fitting 200.

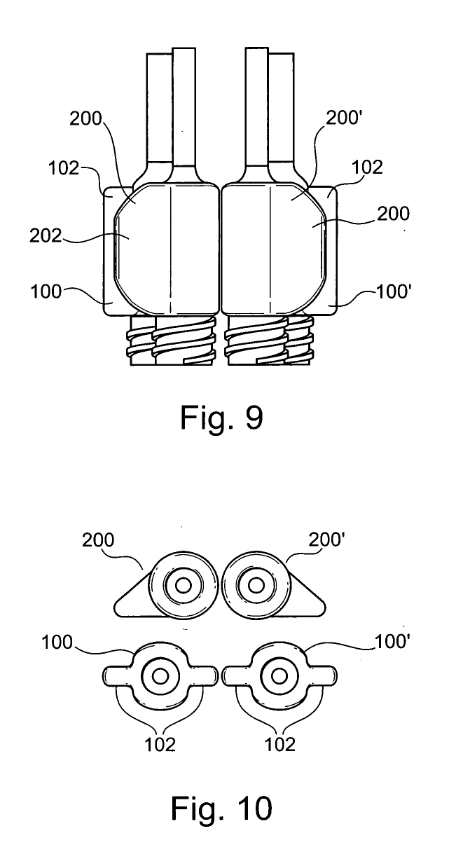

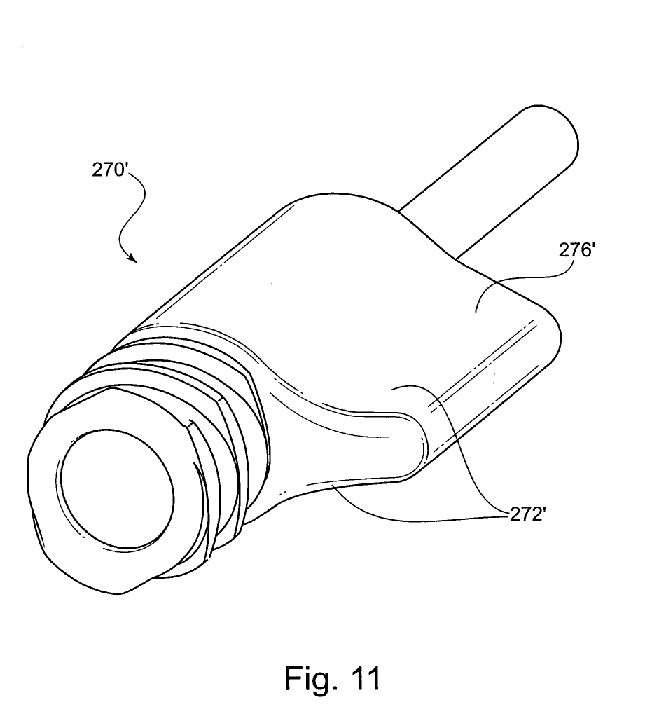

The cross-section of the protrusion 202 of the fitting 200 is preferably substantially symmetrical so that the device may be used in a left hand or a right hand orientation with equal ease. However, those skilled in the art will understand that such symmetry is not necessary and asymmetric designs may provide advantages such as, for example, allowing more ergonomic shapes to be employed. Because only one “wing” is used in the exemplary embodiment, two luer fittings 200, 200′ may be placed side by side as shown in FIG. 4 with a reduced overall width of the two fittings as compared to two conventional luer fittings with dual wings. The reduced footprint of the exemplary fittings 200, 200′ lets two of the devices occupy little more room on the patient's skin than a single conventional device. As shown in FIGS. 9 and 10, the single wing 202 of each of the luer fittings 200, 200′ allows these luer fittings 200, 200′ to be placed closer to one another than is possible with the conventional dual wing fittings 100,100′. For example, in this embodiment, the fittings 200, 200′ may be placed adjacent one another for a total width of 0.925 inches while the total width of the two dual wing fittings 100,100′ is 1.012 inches.

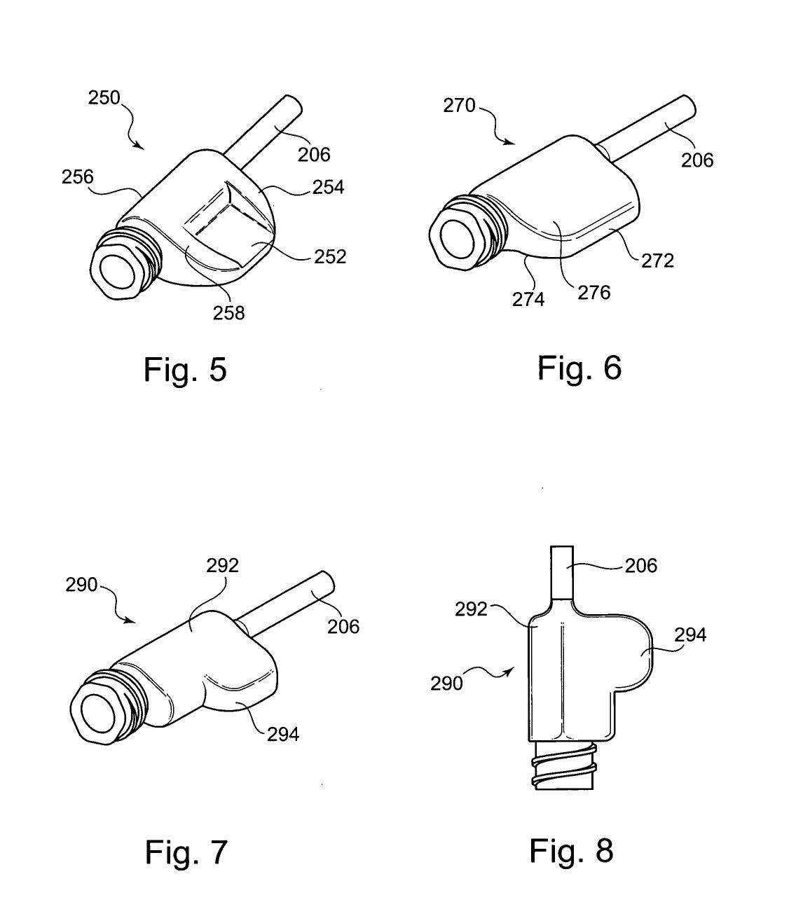

Those of skill in the art will understand that different shapes of the protrusion extending from the luer fittings according to the invention may be used. For example, the radiuses of the curved corners and edges may be varied, as well as the thickness of the flat portions. FIG. 5 shows a different exemplary embodiment of a luer fitting 250 comprising a housing 256 and a protrusion 258. The exemplary protrusion 258 comprises a reduced cross-sectional thickness of the portion 252 with reinforcing structures 254 increasing the structural strength of the protrusion 258 to counteract the effects of the reduced cross-sectional area.

Another exemplary embodiment is shown in FIG. 6. The luer fitting 270 comprises a protrusion 276 having a first concave surface 272 and a second convex surface 274 on a side opposite the surface 272 to provide a better ‘feel’ when applying a large force to twist the connectors. That is, the combination of the concave first surface 272 and the convex second surface 274 provide a more ergonomic form for grasping between the thumb and forefinger. FIG. 11 shows a luer fitting 270′ according to a further embodiment of the invention which is substantially similar to the fitting 270 but with two substantially symmetrical concave surfaces 272′ on opposite sides of the protrusion 276′. As would be understood by those skilled in the art, this arrangement generates a slightly different ergonomic feel and a slightly altered feel when in position against the skin.

Because the protrusion used to rotate the luer fitting according to the invention is large, it is possible to modify its outline to achieve desired visual effects. Letters, symbols, or other characteristic shapes may be obtained by changing the perimeter of the protrusion, while retaining suitability of the fitting for grasping and turning to form the connection. For example, as shown in FIGS. 7 and 8, the luer fitting 290 may comprise a housing 292 with a protrusion 294 that is shaped like the letter “P”. The symbolic shape of the protrusion 294 may indicate, for example, that the luer fitting 290 is suitable for power injection procedures. Those of skill in the art will understand that different letters, numbers and other shapes may be used to convey a variety of messages and/or data.

The luer fittings according to exemplary embodiments of the invention are preferably formed of plastics or other suitable moldable materials. For example, the housing and the protrusion of the fitting may be molded in a conventional procedure to achieve the desired flat, rounded shape. However, as would be understood by those skilled in the art, a variety of other conventional manufacturing techniques may be used to produce the luer fittings according to the invention and different materials may be employed, depending on the manufacturing costs and/or difficulties as well as the properties desired of the finished product.

The present invention has been described with reference to specific embodiments, and more specifically to a luer fitting for power injecting a fluid and to infuse a fluid into PICC. However, other embodiments may be devised that are applicable to other medical devices, without departing from the scope of the invention. Accordingly, various modifications and changes may be made to the embodiments, without departing from the broadest spirit and scope of the present invention as set forth in the claims that follow. The specification and drawings are accordingly to be regarded in an illustrative rather than restrictive sense.

What is claimed is:

1. A fitting for connecting a catheter to a fluid conduit, comprising:
a housing defining a lumen extending therethrough from a distal end adapted for attachment to a catheter and a proximal end releasably connectable to a second fitting; and
a wing extending from the housing away from an axis of the lumen, a portion of the housing opposite the wing being substantially rounded and free of projections away from the axis, a radially outermost lateral edge of a first surface of the wing being rounded wherein, when in a desired position, the first surface faces the skin.

2. The fitting according to claim 1, wherein a thickness of the wing varies in a direction substantially parallel to the axis.

3. The fitting according to claim 2, wherein the wing includes a reduced thickness portion and at least one thicker reinforcing element extending from the housing to the lateral edge.

4. The fitting according to claim 1, wherein a shape of the wing is selected to cooperate with a shape of the housing to depict a symbol.

5. The fitting according to claim 3, wherein the symbol is a letter.

6. The fitting according to claim 1, wherein the wing is substantially symmetrical with respect to a plane including the axis.

7. The fitting according to claim 1, wherein the first surface of the wing is substantially convex and wherein a second surface of the wing opposite the first surface is substantially concave.

8. The fitting according to claim 1, wherein the housing includes a connector suitable for a power injectable catheter.

9. A luer fitting, comprising:
a housing connectable to a power injection catheter;
a wing extending from the housing substantially radially away from a longitudinal axis of a lumen of the housing, a skin contacting surface of the wing being joined to other surfaces of the wing at curved edges; and
a portion of the housing opposite the wing being rounded.

10. The luer fitting according to claim 10, wherein a radius of the curved edges is preferably at least 0.015 inches or more preferably at least 0.030 inches.

11. The luer fitting according to claim 10, wherein the wing comprises a reduced thickness portion and a thicker reinforcing element extending radially away from the housing toward the lateral edge.

12. The luer fitting according to claim 11, wherein the wing comprises first and second reinforcing elements separated from one another by the reduced thickness portion.

13. The luer fitting according to claim 10, wherein a shape of the wing cooperates with a shape of the housing to form a symbol.

14. The luer fitting according to claim 13, wherein the symbol comprises the letter ‘P’.

15. The luer fitting according to claim 10, wherein the wing is substantially symmetrical with respect to a plane including the axis.

16. The luer fitting according to claim 15, wherein a cross-section of the wing in a plane substantially perpendicular to the axis is wedge-shaped.

17. The luer fitting according to claim 10, wherein the connection portion is a connector suitable for attachment to a power injection catheter.
